(19) United States
(12) Patent Application Publication
(10) Pub. No.: US 2025/0019703 A1
(43) Pub. Date: Jan. 16, 2025

(54) PHARMACEUTICAL COMPOSITION FOR PREVENTION OR TREATMENT OF BLOOD CANCER COMPRISING SURF4 INHIBITOR

(71) Applicant: PUSAN NATIONAL UNIVERSITY INDUSTRY-UNIVERSITY COOPERATION FOUNDATION, Busan (KR)

(72) Inventors: Dongjun LEE, Gyeongsangnam-do (KR); Jayoung KIM, Busan (KR)

(21) Appl. No.: 18/711,961

(22) PCT Filed: Jul. 15, 2022
(86) PCT No.: PCT/KR2022/010360

§ 371 Date: May 21, 2024

(30) Foreign Application Priority Data
Dec. 16, 2021 (KR) 10-2021-0180246

(51) Int. Cl. C12N 15/113; A61K 31/337; A61K 31/7072; A61P 35/02

(52) U.S. Cl. CPC C12N 15/113; A61K 31/337; A61K 31/7072; A61P 35/02; C12N 2310/531

(57) ABSTRACT

One aspect relates to a pharmaceutical composition for the prevention or treatment of blood cancer, comprising a surfeit locus protein 4 (SURF4) inhibitor. The composition according to one aspect inhibits SURF4 to increase the expression of pJNK and decrease the expression of pERK and pAKT, and thus apoptosis of blood cancer cells is increased, thereby exhibiting an effect of preventing or treating blood cancers. In addition, by administering the composition according to one aspect together with an existing anticancer agent, a synergistic effect showing more significant apoptosis than when an existing anticancer agent was administered was confirmed, which can contribute to the blood cancer treatment market/industry.

### TECHNICAL FIELD

One aspect of the present invention relates to a pharmaceutical composition for the prevention or treatment of blood cancer including a surfeit locus protein 4 (SURF4) inhibitor.

### BACKGROUND ART

Blood cancer is a comprehensive term for cancers occurring in components constituting blood and refers to malignant tumors occurring in the blood, hematopoietic organs, lymph nodes, or lymphoid organs.

Recently, among blood cancers, the incidence of not only leukemia but also multiple myeloma, lymphoma, myelodysplastic syndrome, myeloproliferative tumors, and the like are rapidly increasing. According to the 2015 National Cancer Registration statistics (Korea Central Cancer Registry of the Ministry of Health and Welfare), blood cancer patients account for about 5.2% of all cancers. When looking at the increase in the number of new patients by major blood cancer type over the past decade, non-Hodgkin's lymphoma increased 89% and for multiple myeloma increased 122%, far outpacing the 51% increase in the overall number of cancer patients over the same period. In addition, the incidence of leukemia appears to be about 10 times higher in adults than in children, and in particular, acute myeloid leukemia (AML) is the most common form of acute leukemia in adults, accounting for 65% of acute leukemias. As such, the incidence of blood cancer is higher in adults, and as aging progresses, the prevalence and mortality rate of blood cancer continue to increase, and it is expected that the incidence of blood cancer will increase up to 10% in the future as Korea's population ages.

Unlike solid cancers, such as liver cancer and lung cancer, in which tumors occur in specific organs, blood cancer has a high risk of metastasis and are difficult to treat because cancer cells travel all throughout the body through the blood. Therefore, to treat blood cancer, chemotherapy is performed in most cases, and bone marrow transplantation, localized radiation therapy, and local surgery are performed in combination. However, anticancer therapies are still limited, and conventional treatment methods have many side effects. In particular, conventional chemotherapy was performed using paclitaxel and tunicamycin as anticancer drugs, but the reality is that there is an absence of a master gene to induce synergistic apoptosis other than these two drugs, and the specific function in anticancer action is unknown.

Accordingly, the present inventors have identified SURF4 as a new therapeutic target for blood cancer and confirmed that blood cancer may be prevented or treated by inhibiting SURF4 expression, and furthermore, there is a synergistic effect on blood cancer treatment with the existing anticancer drugs paclitaxel or tunicamycin, thereby completing the present invention.

### DISCLOSURE

### Technical Problem

One aspect is to provide a pharmaceutical composition for the prevention or treatment of blood cancer, including a surfeit locus protein 4 (SURF4) inhibitor.

Another aspect of the present invention is to provide a health functional food for the prevention or amelioration of blood cancer, including a SURF4 inhibitor.

Still another aspect of the present invention is to provide a method for preventing or treating blood cancer, including administering a SURF4 inhibitor to a subject in need thereof.

Yet another aspect of the present invention is to provide a use of a SURF4 inhibitor for preparing a drug for the prevention or treatment of blood cancer.

### Technical Solution

One aspect is to provide a pharmaceutical composition for the prevention or treatment of blood cancer, including a SURF4 inhibitor.

In one aspect, the SURF4 inhibitor may be at least one selected from the group consisting of a polypeptide, a polynucleotide, and a compound. Specifically, the SURF4 inhibitor may be at least one selected from the group consisting of short hairpin RNA (shRNA), small interfering RNA (siRNA), and micro RNA (miRNA).

The SURF4 inhibitor may be derived from natural sources or may be synthesized according to a conventional method in the art. For example, the SURF4 inhibitor may be prepared using polynucleotide recombination and a protein expression system, or may be prepared by a method of synthesizing in vitro through chemical synthesis, such as peptide synthesis, or by a cell-free protein synthesis method. In addition, as an example, the polypeptide may be a peptide an extract of plant-derived tissues or cells or a product obtained by culturing microorganisms (e.g., bacteria or fungi, and particularly, yeast).

Specifically, when the SURF4 inhibitor is a polynucleotide or a polypeptide, the SURF4 inhibitor may a culture product of cells genetically engineered to express the SURF4 inhibitor.

The term “polynucleotide” refers to deoxyribonucleic acid (DNA) or ribonucleic acid (RNA) in single- or double-stranded form. Unless otherwise limited, it may also include known analogs of naturally occurring nucleotides that hybridize to nucleic acids in a manner similar to naturally occurring nucleotides.

The term “polypeptide” refers to a polymer consisting of two or more amino acids linked by an amide bond (or a peptide bond). The term “expression” includes all functions by which the encoded information of a gene is converted into a structure that is present and operates in cells.

The term “genetic engineering” or “genetically engineered” refers to an act of introducing one or more genetic modifications to cells, or cells created thereby.

Specifically, the cells genetically engineered to express the SURF4 inhibitor may be cells transfected with a vector encoding a gene expressing the SURF4 inhibitor.

More specifically, the genetic engineering may exhibit an effect of preventing or treating blood cancer through a process of transfecting cells with a virus for transformation including a vector including a gene encoding the SURF4 inhibitor, administering to blood cancer cells or treating blood cancer cells with a cell culture including the virus for transformation expressed by the cells, and inducing apoptosis of blood cancer cells by the expression of the SURF4 inhibitor. “Vector” refers to a means for expressing a target gene in a host cell. For example, it includes plasmid vectors, cosmid vectors, and viral vectors such as bacteriophage vectors, adenovirus vectors, retrovirus vectors, and adeno-associated virus vectors. Further, the viruses for transformation include, for example, adenoviruses, retroviruses, adeno-associated viruses, or lentiviruses.

The term “transformation” refers to the change in the genetic characteristics of an living organism by externally provided DNA, and it means introducing a gene into a host cell so that the gene may be expressed in the host cell, and the term “transfection” refers to a technique of implanting externally provided DNA into a cell, and it is a phenomenon in which, when DNA, a type of nucleic acid extracted from a cell of a certain strain of living organism (excluding viral cells), is transfected into a living cell of another strain, the DNA enters the cell and changes its genetic characteristics. In other words, it means introducing a specific gene into a host cell so that the gene may be expressed in the host cell.

The term “cell culture” refers to a population of cells suspended in a medium under conditions suitable for the survival and/or proliferation of the population of cells. In addition, it also refers to a mixture containing a population of cells and a medium in which the population is suspended. The cell culture may be cultured using a method known in the art, and the cell culture may be appropriately supplemented with an additive necessary for the survival, culture, etc. of target cells.

The term “medium” refers to a nutrient-containing solution that nourishes proliferating cells. Such a solution typically provides essential and non-essential amino acids, vitamins, energy sources, lipids, and trace elements necessary for cells to minimally proliferate and/or survive. In addition, this solution may further contain ingredients that enhance proliferation and/or survival beyond a minimum rate, including hormones and growth factors. This solution is preferably formulated at a pH and a salt concentration that are optimal for cell survival and proliferation. The medium may also be a “synthetic medium,” that is, a serum-free medium that is completely free of ingredients, proteins, or hydrolyzates of an unknown composition. A synthetic medium is a medium which is free of any ingredients of animal origin and in which all ingredients have a known chemical structure.

In one aspect, the SURF4 inhibitor may increase reactive oxygen species (ROS) in blood cancer cells. For example, the SURF4 inhibitor may increase at least one selected from the group consisting of hydrogen peroxide, superoxide anions, hydroxyl radicals, lipid peroxides, nitric oxide, and peroxynitrites in blood cancer cells.

In one aspect, the SURF4 inhibitor may increase the expression of phosphorylated signal transducer and activator of transcription 6 (pSTAT6) of blood cancer cells. Specifically, the SURF4 inhibitor may act synergistically with IL4 that increases the expression of pSTAT6 in blood cancer cells to further increase the expression of pSTAT6, thereby more significantly increasing apoptosis of blood cancer cells.

In one aspect, the SURF4 inhibitor may regulate the amount of phosphorylated proteins in blood cancer cells. Specifically, the SURF4 inhibitor may increase the expression of cell necrosis regulators in blood cancer cells and decrease the expression of regulatory factors enhancing cell growth and proliferation in blood cancer cells, and more specifically, the SURF4 inhibitor may increase the expression of phosphorylated c-Jun N-terminal kinase (pJNK), a cell necrosis regulator, in blood cancer cells and decrease the expression of phosphorylated extracellular regulated kinase (pERK) and phosphorylated serine/threonine protein kinase B (pAKT), which are regulatory factors enhancing cell growth and proliferation in blood cancer cells. As the expression of the cell necrosis regulator in the blood cancer cells is increased, apoptosis of blood cancer cells may be increased, and as the expression of the regulatory factors enhancing cell growth and proliferation in blood cancer cells is decreased, the cell growth of the blood cancer cells may be decreased.

In one aspect, the blood cancer may be at least one selected from the group consisting of acute myeloid leukemia (AML), chronic myeloid leukemia (CML), Abelson oncogene-associated CML (Bcr-ABL translocation), myelodysplastic syndrome (MDS), B-cell acute lymphoblastic leukemia (B-ALL), T-cell acute lymphoblastic leukemia (T-ALL), chronic lymphocytic leukemia (CLL), multiple myeloma (MM), myeloproliferative neoplasms (MPN), Richter syndrome, Richter transformation of CLL, hairy cell leukemia (HCL), blastic plasmacytoid dendritic cell neoplasm (BPDCN), non-Hodgkin's lymphoma (NHL), mantle cell lymphoma (MCL), small lymphocytic lymphoma (SLL), Hodgkin's lymphoma, systemic mastocytosis, and Burkitt lymphoma. More specifically, the blood cancer may be at least one selected from the group consisting of AML, CML, B-ALL, and T-ALL, and more specifically, it may be at least one selected from the group consisting of AML and CML.

The term “cancer” refers to a class of diseases characterized by the development of abnormal cells that uncontrollably proliferate and have the ability to invade and destroy normal body tissues.

The term “blood cancer” is a word that comprehensively refers to cancers generated in components that make up blood, and it refers to malignancies generated in the blood, hematopoietic organs, lymph nodes, and lymphatic organs.

In addition, in one aspect, the SURF4 inhibitors may not exhibit a preventive or therapeutic effect on solid cancer. Specifically, a culture of cells genetically engineered to express the SURF4 inhibitor may not induce apoptosis of solid cancer cells, unlike blood cancer cells.

The term “solid cancer” refers to a cancer that has features that distinguish it from blood cancer and is made of a mass generated by abnormal cell growth in various solid organs, such as the bladder, breasts, intestines, kidneys, lungs, liver, brain, esophagus, gallbladder, ovaries, pancreas, stomach, cervix, thyroid, prostate, and skin, and the solid cancer may be, for example, at least one selected from the group consisting colon cancer, colorectal cancer, lung cancer, liver cancer, stomach cancer, esophageal cancer, pancreatic cancer, gallbladder cancer, kidney cancer, bladder cancer, prostate cancer, testicular cancer, cervical cancer, endometrial cancer, choriocarcinoma, ovarian cancer, breast cancer, thyroid cancer, brain cancer, head and neck cancer, and malignant melanoma, and more specifically, the solid cancer may be at least one selected from the group consisting of prostate cancer, cervical cancer, ovarian cancer, breast cancer, and colorectal cancer.

The term “prevention” may refer to any act of inhibiting or delaying the onset of blood cancer in a subject by the administration of a pharmaceutical composition according to one aspect.

The term “treatment” may refer to any act of ameliorating or beneficially changing the symptoms of blood cancer in an individual by the administration of a pharmaceutical composition according to one aspect.

The term “administration” refers to introducing a predetermined substance to a subject by an appropriate method, and the term “subject” refers to any living organism that may have blood cancer, including humans as well as rats, mice, livestock, etc. As a specific example, it may be a mammal, including a human.

In addition, the pharmaceutical composition may be provided as a pharmaceutical composition including an active ingredient alone or including one or more pharmaceutically acceptable carriers, excipients, or diluents.

Specifically, the carriers may be, for example, colloidal suspensions, powder, saline solutions, lipids, liposomes, microspheres, or nano-spherical particles. They may form a complex with or be associated with a delivery means and may be transported in vivo using any delivery system known in the art, such as lipids, liposomes, microparticles, gold, nanoparticles, polymers, condensation reaction agents, polysaccharides, poly(amino acids), dendrimers, saponins, adsorption enhancers, or fatty acids.

When the pharmaceutical composition is formulated, it may be formulated using commonly used diluents or excipients such as lubricants, sweeteners, flavoring agents, emulsifiers, suspending agents, preservatives, filling agents, extending agents, binders, wetting agents, disintegrating agents, surfactants, and the like. Solid formulations for oral administration may include tablets, pills, powder, granules, capsules, and the like, and these solid formulations may be formulated by mixing the composition with at least one excipient, such as starch, calcium carbonate, sucrose or lactose, gelatin, and the like. In addition to simple excipients, lubricants such as magnesium stearate and talc may also be used. Liquid formulations for oral administration include suspensions, oral liquids, emulsions, and syrups, and may contain various excipients, such as wetting agents, sweeteners, fragrances, and preservatives, in addition to water and liquid paraffin, which are commonly used simple diluents. Formulations for parenteral administration may include sterile aqueous solutions, non-aqueous solvents, suspensions, emulsions, lyophilized preparations, and suppositories. Non-aqueous solvents and suspensions may include propylene glycol, polyethylene glycol, vegetable oils such as olive oil, and injectable esters such as ethyl oleate. As a base of suppositories, Witepsol, Macrogol, Tween 61, cacao oil, laurin oil, glycerol gelatin, and the like may be used, and when the pharmaceutical composition is prepared in the form of eye drops, known diluents or excipients may be used.

In one aspect, the pharmaceutical composition may further include an anticancer agent.

The pharmaceutical composition may be provided in combination with a conventionally known composition for the prevention or treatment of cancer or another conventional anticancer agent, and the other anticancer agent may be a conventionally known composition for the prevention or treatment of cancer, an existing anticancer agent, or a newly developed anticancer agent.

When the pharmaceutical composition further includes an anticancer agent, it is important that the anticancer agent is mixed in an amount that can achieve the maximum effect with the minimum amount without side effects, which may be easily determined by those skilled in the art.

In one aspect, the anticancer agent may be, for example, at least one selected from the group consisting of paclitaxel and tunicamycin.

When the pharmaceutical composition further includes an anticancer agent, the SURF4 inhibitor may further increase the expression of an apoptosis-related protein and/or an endoplasmic reticulum stress-related gene in blood cancer cells compared to a case in which the anticancer agent is administered alone.

Specifically, when the pharmaceutical composition further includes an anticancer agent, and the anticancer agent includes paclitaxel, the SURF4 inhibitor may further increase the expression of an apoptosis-related protein in blood cancer cells compared to a case in which paclitaxel is administered alone. In addition, when the pharmaceutical composition further includes an anticancer agent, and the anticancer agent includes tunicamycin, the SURF4 inhibitor may further increase the expression of an endoplasmic reticulum stress-related gene in blood cancer cells compared to a case in which tunicamycin is administered alone.

More specifically, when the pharmaceutical composition further includes an anticancer agent, and the anticancer agent includes paclitaxel, the SURF4 inhibitor may further increase the apoptosis of blood cancer cells by increasing the expression of at least one protein selected from the group consisting of cleaved caspase 9, cleaved caspase 3, and poly(adenosine diphosphate-ribose) polymerase (PARP) in blood cancer cells. In addition, when the pharmaceutical composition further includes an anticancer agent, and the anticancer agent includes tunicamycin, the SURF4 inhibitor may further increase the apoptosis of blood cancer cells by increasing the expression of at least one gene selected from the group consisting of protein kinase R-like endoplasmic reticulum kinase (PERK), inositol requiring kinase 1a (pelF2α), eukaryotic translation initiation factor 2α (elF2α), activating transcription factor 4 (ATF4), and CCAAT/enhancer-binding protein homologous protein (CHOP) in blood cancer cells.

In addition, in one aspect, the pharmaceutical composition may be administered alone or in combination with the anticancer agent.

The pharmaceutical composition may be administered in combination with a known composition having an effect of preventing or treating cancer or another anticancer agent, and may be administered concomitantly, separately, or sequentially, and may be administered in a single dose or in multiple doses. It is important to consider all of the above factors and administer an amount that can achieve the maximum effect with the minimum amount, which may be easily determined by those skilled in the art.

In one aspect, the anticancer agent may be, for example, at least one selected from the group consisting of paclitaxel and tunicamycin.

When the pharmaceutical composition is administered in combination with an anticancer agent, the SURF4 inhibitor may further increase the expression of an apoptosis-related protein and/or an endoplasmic reticulum stress-related gene in blood cancer cells compared to a case in which the anticancer agent is administered alone.

Specifically, when the pharmaceutical composition is administered in combination with an anticancer agent, and the anticancer agent includes paclitaxel, the SURF4 inhibitor may further increase the expression of an apoptosis-related protein in blood cancer cells compared to a case in which paclitaxel is administered alone. In addition, when the pharmaceutical composition is administered in combination with an anticancer agent, and the anticancer agent includes tunicamycin, the SURF4 inhibitor may further increase the expression of an endoplasmic reticulum stress-related gene in blood cancer cells compared to a case in which tunicamycin is administered alone.

More specifically, when the pharmaceutical composition is administered in combination with an anticancer agent, and the anticancer agent includes paclitaxel, the SURF4 inhibitor may further increase the apoptosis of blood cancer cells by increasing the expression of at least one protein selected from the group consisting of cleaved caspase 9, cleaved caspase 3, and PARP in blood cancer cells. In addition, when the pharmaceutical composition further includes an anticancer agent, and the anticancer agent includes tunicamycin, the SURF4 inhibitor may further increase the apoptosis of blood cancer cells by increasing the expression of at least one gene selected from the group consisting of PERK, pelF2α, elF2α, ATF4, and CHOP in blood cancer cells.

The pharmaceutical composition may be administered orally or parenterally, and when administered parenterally, external skin application or intraperitoneal injection, rectal injection, subcutaneous injection, intravenous injection, intramuscular injection, intraarterial injection, intramedullary injection, intracardiac injection, intrathecal injection, transdermal injection, intranasal injection, enteral injection, topical injection, sublingual injection, rectal injection, or intrathoracic injection method may be selected.

The pharmaceutical composition is administered in a pharmaceutically effective amount. The term, “pharmaceutically effective amount” refers to an amount sufficient to treat a disease at a reasonable benefit/risk ratio applicable to medical treatment, and an effective dose level may be determined based on factors including the type and severity of the patient's disease, the activity of the drug, sensitivity to the drug, time of administration, route of administration and rate of elimination, duration of treatment, and concomitant medications, and other factors well known in the medical field.

In one aspect, when the anticancer agent is paclitaxel, the anticancer agent may be administered at a concentration of 0.1 to 1.0 μM, 0.1 to 0.8 μM, 0.1 to 0.6 μM, 0.2 to 1.0 μM, 0.2 to 0.8 μM, 0.2 to 0.6 μM, 0.4 to 1.0 μM, 0.4 μM to 0.8 μM, or 0.4 to 0.6 μM.

When the anticancer agent is administered at a concentration of less than 0.1 μM, the apoptotic effect on blood cancer cells may be reduced, and when administered at a concentration of higher than 1.0 μM, the apoptosis of normal cells other than blood cancer cells may be enhanced.

In one aspect, where the anticancer agent is tunicamycin, the anticancer agent may be administered at 1 to 20 μg/ml, 1 to 15 μg/ml, 1 to 13 μg/ml, 5 to 20 μg/ml, 5 to 15 μg/ml, 5 to 13 μg/ml, 7 to 20 μg/ml, 7 to 15 μg/ml, or 7 to 13 μg/ml.

When the anticancer agent is administered at a concentration of less than 1 μg/ml, the apoptotic effect on blood cancer cells may be reduced, and when administered at a concentration of higher than 20 μg/ml, the apoptosis of normal cells other than blood cancer cells may be enhanced.

In one aspect, the anticancer agent may be administered 3 to 14 days, 3 to 12 days, 3 to 11 days, 5 to 14 days, 5 to 12 days, 5 to 11 days, 6 to 14 days, 6 to 12 days, or 6 to 11 days after the administration of the SURF4 inhibitor.

When the anticancer agent is administered less than 3 days or more than 14 days after the administration of the SURF4 inhibitor, there may not be a synergistic effect on the prevention or treatment of blood cancer to exhibit excellent apoptosis compared to a case in which the anticancer agent is administered 7 to 10 days after the administration of the SURF4 inhibitor.

In one aspect, the pharmaceutical composition may be administered once daily, or may be administered in several divided doses. For example, it may be administered every second day, or it may be administered once a week.

Another aspect of the present invention provides a health functional food for the prevention or amelioration of blood cancer, including a SURF4 inhibitor.

The “SURF4 inhibitor,” “blood cancer,” “prevention,” and the like may be as described above.

The term “amelioration” may refer to any act that at least reduces a parameter related to the condition being treated, for example, the severity of symptoms. At this time, the health functional food may be used for the prevention or amelioration of blood cancer before or after the onset of the disease, concomitantly with or independently of a drug for treatment.

In the health functional food, an active ingredient may be added to the food as is or used in combination with other foods or food ingredients, and may be appropriately used according to conventional methods. The mixing amount of the active ingredient may be suitably determined according to the purpose of use (for prevention or amelioration). In general, when a food or beverage is prepared, the health functional food may be added in an amount of about 15% by weight or less based on raw materials, more specifically about 10% by weight or less. However, in the case of long-term intake for the purpose of health and hygiene or health control, the amount may be below the above range.

The health functional food may be formulated as one selected from the group consisting of tablets, pills, powder, granules, powder, capsules, and liquid dosage forms by further including one or more of carriers, diluents, excipients, and additives. Food to which a compound according to one aspect may be added includes various food products, powder, granules, tablets, capsules, syrups, beverages, chewing gum, teas, vitamin complexes, health functional food, and the like.

Specific examples of the above carriers, excipients, diluents, and additives may be at least one selected from the group consisting of lactose, dextrose, sucrose, sorbitol, mannitol, erythritol, starch, acacia gum, calcium phosphate, alginate, gelatin, calcium phosphate, calcium silicate, microcrystalline cellulose, polyvinylpyrrolidone, cellulose, polyvinylpyrrolidone, methylcellulose, water, sugar syrup, methylcellulose, methyl hydroxybenzoate, propyl hydroxybenzoate, talc, magnesium stearate, and mineral oil.

In addition to containing the above active ingredients, the health functional food may contain other ingredients as essential ingredients without limitation. For example, like conventional beverages, it may contain various flavoring agents or natural carbohydrates as additional ingredients. Examples of the above-mentioned natural carbohydrates may be monosaccharides, for example, glucose, fructose, and the like; disaccharides, for example, maltose, sucrose, and the like; and polysaccharides, for example, conventional sugars such as dextrin, cyclodextrin, and the like, and sugar alcohols such as xylitol, sorbitol, erythritol, and the like. In addition to what is described above, as flavoring agents, natural flavoring agents (such as thaumatin, stevia extract (for example, rebaudioside A, glycyrrhizin, and the like)) and synthetic flavoring agents (such as saccharin, aspartame, and the like) may be advantageously used. The proportions of the natural carbohydrates may be appropriately selected by those skilled in the art.

In addition to what is described above, the health functional food according to one aspect may contain various nutrients, vitamins, minerals (electrolytes), flavoring agents such as synthetic flavoring agents and natural flavoring agents, coloring agents and enhancers (cheese, chocolate, or the like), pectic acid and salts thereof, alginic acid and salts thereof, organic acids, protective colloidal thickeners, pH regulators, stabilizers, preservatives, glycerin, alcohol, carbonation agents used in carbonated beverages, and the like. These ingredients may be used independently or in combination, and the proportions of these additives may be appropriately selected by those skilled in the art.

In one aspect, the health functional food may further include a health functional food for the prevention or amelioration of cancer.

The health functional food may be provided in combination with a conventionally known health functional food for the prevention or amelioration of cancer or a newly developed health functional food for the prevention or amelioration of cancer.

When the health functional food further includes a health functional food for the prevention or amelioration of cancer, it is important to mix an amount that can achieve the maximum effect with the minimum amount without side effects, which may be easily determined by those skilled in the art.

In one aspect, the health functional food for the prevention or amelioration of cancer may be a health functional food for the prevention or amelioration of cancer including, for example, at least one selected from the group consisting of paclitaxel and tunicamycin.

When the health functional food further includes a health functional food for the prevention or amelioration of cancer, the SURF4 inhibitor may further increase the expression of an apoptosis-related protein and/or an endoplasmic reticulum stress-related gene in blood cancer cells compared to a case in which the health functional food for the prevention or amelioration of cancer is administered alone.

Specifically, when the health functional food further includes a health functional food for the prevention or amelioration of cancer, and the health functional food for the prevention or amelioration of cancer includes paclitaxel, the SURF4 inhibitor may further increase the expression of an apoptosis-related protein in blood cancer cells compared to a case in which the health functional food for the prevention or amelioration of cancer is administered alone. In addition, when the health functional food further includes a health functional food for the prevention or amelioration of cancer, and the health functional food for the prevention or amelioration of cancer includes tunicamycin, the SURF4 inhibitor may further increase the expression of an endoplasmic reticulum stress-related gene in blood cancer cells compared to a case in which the health functional food for the prevention or amelioration of cancer is administered alone.

More specifically, when the health functional food further includes a health functional food for the prevention or amelioration of cancer, and the health functional food for the prevention or amelioration of cancer includes paclitaxel, the SURF4 inhibitor may further increase the apoptosis of blood cancer cells by increasing the expression of at least one protein selected from the group consisting of cleaved caspase 9, cleaved caspase 3, and PARP in blood cancer cells. In addition, when the health functional food further includes a health functional food for the prevention or amelioration of cancer, and the health functional food for the prevention or amelioration of cancer includes tunicamycin, the SURF4 inhibitor may further increase the apoptosis of blood cancer cells by increasing the expression of at least one gene selected from the group consisting of PERK, pelF2α, elF2α, ATF4, and CHOP in blood cancer cells.

In addition, in one aspect, the health functional food may be administered alone or in combination with the health functional food for the prevention or amelioration of cancer.

The health functional food may be administered in combination with a known composition or another health functional food for the prevention or amelioration of cancer having an effect of preventing or treating cancer, and may be administered concomitantly, separately, or sequentially, and may be administered in a single dose or in multiple doses. It is important to consider all of the above factors and administer an amount that can achieve the maximum effect with the minimum amount without side effects, which may be easily determined by those skilled in the art.

In one aspect, the health functional food for the prevention or amelioration of cancer may be a health functional food for the prevention or amelioration of cancer including, for example, at least one selected from the group consisting of paclitaxel and tunicamycin.

When the health functional food is ingested in combination with a health functional food for the prevention or amelioration of cancer, the SURF4 inhibitor may further increase the expression of an apoptosis-related protein and/or an endoplasmic reticulum stress-related gene in blood cancer cells compared to a case in which the health functional food for the prevention or amelioration of cancer is administered alone.

Specifically, when the health functional food is ingested in combination with a health functional food for the prevention or amelioration of cancer, and the health functional food for the prevention or amelioration of cancer includes paclitaxel, the SURF4 inhibitor may further increase the expression of an apoptosis-related protein in blood cancer cells compared to a case in which the health functional food for the prevention or amelioration of cancer is administered alone. In addition, when the health functional food is ingested in combination with a health functional food for the prevention or amelioration of cancer, and the health functional food for the prevention or amelioration of cancer includes tunicamycin, the SURF4 inhibitor may further increase the expression of an endoplasmic reticulum stress-related gene in blood cancer cells compared to a case in which the health functional food for the prevention or amelioration of cancer is administered alone.

More specifically, when the health functional food is ingested in combination with a health functional food for the prevention or amelioration of cancer, and the health functional food for the prevention or amelioration of cancer includes paclitaxel, the SURF4 inhibitor may further increase the apoptosis of blood cancer cells by increasing the expression of at least one protein selected from the group consisting of cleaved caspase 9, cleaved caspase 3, and PARP in blood cancer cells. In addition, when the health functional food is ingested in combination with a health functional food for the prevention or amelioration of cancer, and the health functional food for the prevention or amelioration of cancer includes tunicamycin, the SURF4 inhibitor may further increase the apoptosis of blood cancer cells by increasing the expression of at least one gene selected from the group consisting of PERK, pelF2α, elF2α, ATF4, and CHOP in blood cancer cells.

Another aspect provides a method for preventing or treating blood cancer, including a step of administering a SURF4 inhibitor to a subject in need thereof.

The “SURF4 inhibitor,” “subject,” “administration,” “blood cancer,” “prevention,” “treatment,” and the like may be as described above.

In addition, in one aspect, the method may involve administering the SURF4 inhibitor to a subject in need thereof, alone or in combination with an anticancer agent.

The method may involve administering a known composition having an effect of preventing or treating cancer or another anticancer agent in combination, and may be involve administering concomitantly, separately, or sequentially, and may involve administering in a single dose or in multiple doses. It is important to consider all of the above factors and administer an amount that can achieve the maximum effect with the minimum amount without side effects, which may be easily determined by those skilled in the art.

In one aspect, the anticancer agent may be, for example, at least one selected from the group consisting of paclitaxel and tunicamycin.

When the method involves administering an anticancer agent in combination, the SURF4 inhibitor may further increase the expression of an apoptosis-related protein and/or an endoplasmic reticulum stress-related gene in blood cancer cells compared to a case in which the anticancer agent is administered alone.

Specifically, when the method involves administering an anticancer agent in combination, and the anticancer agent includes paclitaxel, the SURF4 inhibitor may further increase the expression of an apoptosis-related protein in blood cancer cells compared to a case in which the anticancer agent is administered alone. In addition, when the method involves administering an anticancer agent in combination, and the anticancer agent includes tunicamycin, the SURF4 inhibitor may further increase the expression of an endoplasmic reticulum stress-related gene in blood cancer cells compared to a case in which the anticancer agent is administered alone.

More specifically, when the method involves administering an anticancer agent in combination, and the anticancer agent includes paclitaxel, the SURF4 inhibitor may further increase the apoptosis of blood cancer cells by increasing the expression of at least one protein selected from the group consisting of cleaved caspase 9, cleaved caspase 3, and PARP in blood cancer cells. In addition, when the method involves administering an anticancer agent in combination, and the anticancer agent includes tunicamycin, the SURF4 inhibitor may further increase the apoptosis of blood cancer cells by increasing the expression of at least one gene selected from the group consisting of PERK, pelF2α, elF2α, ATF4, and CHOP in blood cancer cells.

Another aspect provides a use of a SURF4 inhibitor for preparing a drug for the prevention or treatment of blood cancer.

The “SURF4 inhibitor,” “blood cancer,” “prevention,” “treatment,” and the like may be as described.

### Advantageous Effects

According to one aspect, it was confirmed that when SURF4 is inhibited, the expression of pJNK is increased, and the expression of pERK and pAKT is decreased, thereby increasing the apoptosis of blood cancer cells. In addition, when SURF4 is inhibited together with the administration of an existing anticancer agent, a synergistic effect showing more significant apoptosis than when an existing anticancer agent was administered was confirmed, which can contribute to the blood cancer treatment market/industry.

### DESCRIPTION OF DRAWINGS

FIG. 1 shows the results of a quantitative real-time polymerase chain reaction (qRT-PCR) analysis for confirming SURF4 gene expression in THP1, human leukemia 60 (HL60), and K562 cell lines transfected with SURF4 shRNA (error bars indicate the standard error of the mean (** p≤0.01)).

FIG. 2 shows the number and apoptosis measurements of THP1, HL60, and K562 cells transfected with SURF4 shRNA (error bars indicate the standard error of the mean (** p≤0.01)).

FIG. 3 shows the confirmation of the expression of pJNK, JNK, and bcl2-associated X protein (BAX) in THP1 cells transfected with SURF4 shRNA using Western blot (left) and a qRT-PCR analysis to confirm the expression of SURF4 gene in THP1 cell line transfected with SURF4 shRNA (right) (error bars indicate the standard error of the mean (** p≤0.01)).

FIGS. 4A, 4B and 4C show the measurement of a normalized fold change in the mean fluorescence intensity (MFI) for phosphorylated proteins in HL60 and THP1 cells, specifically, FIGS. 4A and 4C show the measurement of a normalized fold change in MFI for phosphorylated proteins in HL60 and THP1 cells transfected with SURF4 shRNA, and FIG. 4B shows the extent of apoptosis in THP1, HL60 and K562 cells transfected with SURF4 shRNA via fluorescence activated cell sorting (FACS) (error bars indicate the standard error of the mean (** p≤0. 01)).

FIGS. 5A and 5B show the results of measuring apoptosis after treating SURF4 shRNA-transfected myeloid leukemia cells with paclitaxel, specifically, FIG. 5A shows the results of measuring the apoptosis of THP1 cells, FIG. 5B shows the results of measuring the apoptosis of HL60 cells, and FIG. 5C shows the results of measuring the apoptosis of K562 cells (error bars indicate the standard error of the mean (** p≤0.01)).

FIG. 6 shows the results of confirming the expression of caspase 3, cleaved caspase 3, and PARP in THP1 cells transfected with SURF4 shRNA by Western blot (error bars indicate the standard error of the mean (** p≤0.01)).

FIGS. 7A, 7B and 7C show the results of measuring apoptosis after treating SURF4 shRNA-transfected myeloid leukemia cells with tunicamycin, specifically, FIG. 7A shows the results of measuring the apoptosis of THP1 cells, FIG. 7B shows the results of measuring the apoptosis of HL60 cells, and FIG. 7C shows the results of measuring the apoptosis of K562 cells (error bars indicate the standard error of the mean (** p≤0.01)).

FIG. 8 shows the results of confirming the expression of PERK, pelF2α, elF2α, ATF4, and CHOP in THP1 cells transfected with SURF4 shRNA by Western blot and confirming the expression of CHOP by qRT-PCR (error bars indicate the standard error of the mean) (** p≤0.01)).

FIG. 9 shows the results of confirming the growth inhibition of myeloid leukemia cells transfected with SURF4 shRNA, and specifically, it shows the results of measuring a normalized fold change quantification of MFI for myeloid differentiation (CD11b+) (error bars indicate the standard error of the mean (** p≤0.01)).

FIG. 10 shows the results of confirming the growth inhibition of myeloid leukemia cells transfected with SURF4 shRNA, and specifically, it shows the results of measuring a normalized fold change quantification of MFI for reactive oxygen species (ROS) (error bars indicate the standard error of the mean (** p≤0.01)).

FIG. 11 shows the results of confirming the growth inhibition of myeloid leukemia cells transfected with SURF4 shRNA, and specifically, it shows the results of confirming tumor development in a xenograft model using nonobese diabetic-severe combined immunodeficiency (NOD-SCID) mice (error bars indicate the standard error of the mean (** p≤0.01)).

FIG. 12 shows the interleukin-4-signal transducer and activator of transcription 6 (IL4-STAT6) and stimulator of interferon genes (STING)-dependent apoptosis in myeloid leukemia cells transfected with SURF4 shRNA, and specifically, it shows the results of confirming the expression of phosphorylated signal transducer and activator of transcription 6 (pSTAT6) after treating the myeloid leukemia cells with L4, which induces the expression of pSTAT6, which induces apoptosis (error bars indicate the standard error of the mean (** p≤0.01)).

FIG. 13 shows the results of measuring the normalized fold change quantification of MFI for pSTAT6 in MII-Af9 leukemia cells of mice transfected with SURF4 sgRNA (error bars indicate the standard error of the mean (** p≤0.01)).

FIG. 14 shows the results of measuring apoptosis after IL4 treatment in THP1 and HL60 cells transfected with SURF4 shRNA, and specifically, FIG. 14A shows THP1 cells, and FIG. 14B shows HL60 cells (error bars indicate the standard error of the mean (** p≤0.01)).

FIGS. 15A and 15B show the IL4-STAT6 and STING-dependent apoptosis in myeloid leukemia cells transfected with SURF4 shRNA, and specifically, FIG. 15A shows the results of the apoptosis in myeloid leukemia cells transfected with SURF4 shRNA, and FIG. 15B shows the results of the apoptosis and expression of phosphorylated TANK-binding kinase 1 (pTBK1) after treatment with cyclic guanosine monophosphate-adenosine monophosphate (cGAMP) (error bars indicate the standard error of the mean (** p≤0.01)).

FIG. 16 is a schematic diagram showing that SURF4 regulates the functions of STAT6 and STING and inhibits the differentiation and apoptosis of myeloid leukemia cells.

FIG. 17 shows the difference in apoptosis according to the expression of SURF4 in solid cancer cells, and specifically, it shows the number of prostate cancer 3 (PC3) and HeLa cells transfected with SURF4 shRNA (error bars indicate the standard error of the mean (** p≤0.01)).

FIGS. 18A, 18B and 18C show the difference in apoptosis according to the expression of SURF4 in solid cancer cells, and specifically, FIG. 18A shows the results of measuring the apoptosis in A2780 cells transfected with SURF4 shRNA, and FIG. 18B shows the results of confirming the expression of pJNK and JNK in in A2780, DLD1, PC3, MCF7, and HCT116 cells transfected with a HA-SURF4 overexpression vector by Western blot, and FIG. 18C shows the expression of inositol-requiring enzyme type 1 (IRE1), PERK, and glucose-regulated protein 78 (GRP78) in THP1 cells transfected with SURF4 shRNA by Western blot (error bars indicate the standard error of the mean (** p≤0.01)).

### MODES OF THE INVENTION

Hereinafter, one or more embodiments will be described in more detail through examples. However, these examples are for illustrative purposes only and the scope of the present disclosure is not limited to these examples.

### EXAMPLES

### Example 1: Growth Inhibition of Myeloid Leukemia Cell Lines by SURF4 shRNA

(1) Confirmation of SURF4 Gene Expression in THP1, HL60, and K562 Cell Lines Transfected with SURF4 shRNA

The SURF4 gene expression in myeloid leukemia cell lines transfected with SURF4 shRNA was confirmed. Specifically, THP1 (human monocyte cell line), HL60 (acute myeloid leukemia cell line), and K562 (chronic myeloid leukemia cell line) cell lines were cultured using an RPMI1640 medium, and 10% FBS and 1% penicillin/streptomycin was added to each medium. More specifically, after transfecting 6 μg of SURF4 shRNA plasmid (Santa Cruz Biotechnology Inc., Cat. No.sc-92607-SH) into 293T cells, a cell culture solution containing the lentivirus expressed by the 293T cells was injected into the THP1, HL60, and K562 cell culture solutions. Then, to confirm the SURF4 gene expression in each transfected cell line, RNA was extracted from the cells using an RNA extraction kit, and cDNA was synthesized from the RNA using a cDNA synthesis kit. A qRT-PCR analysis was performed using the synthesized cDNA, SURF4 primers, and SYBR green.

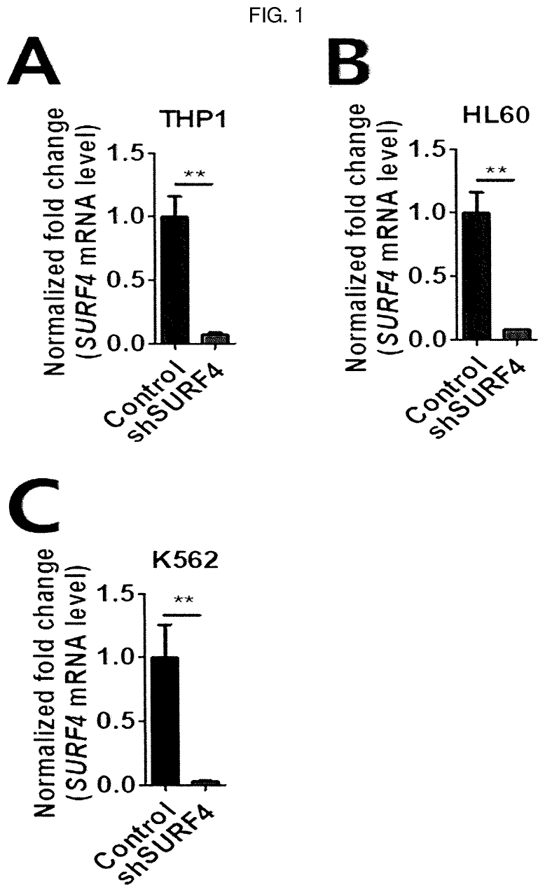

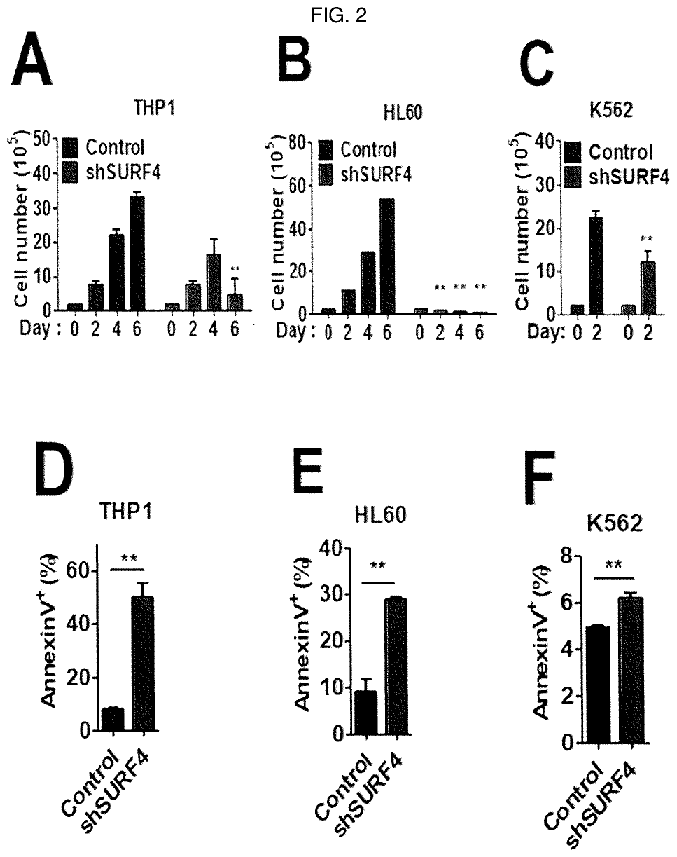

As a result, the SURF4 gene expression in the myeloid leukemia cell lines transfected with SURF4 shRNA was reduced about two-fold compared to the control group (FIG. 1A: THP1 cells, FIG. 1B: HL60 cells, FIG. 1C: K562 cells). In addition, as a result of measuring the number and apoptosis of cells transfected with SURF4 shRNA at two-day intervals, it was confirmed that when the expression of SURF4 was reduced by SURF4 shRNA, cell growth decreased and apoptosis increased, compared to the control group (FIG. 2). Through this, it could be seen that decreased SURF4 expression inhibits the growth of leukemia cells and induces apoptosis thereof.

(2) Confirmation of Protein Amount in THP1 Cells Transfected with SURF4 shRNA

Western blot was performed to confirm the amount of proteins in THP1 cells transfected with SURF4 shRNA. The transfected SURF4 shRNA was the same as in Example 1-(1). Specifically, cells were lysed with a lysis buffer to extract proteins, which were then separated on a 10% SDS-PAGE gel and transferred to a membrane. pJNK, JNK, BAX, caspase 9, cleaved caspase 9, caspase 3, cleaved caspase 3, PARP, IRE1a, GRP78, PERK, pelF2α, elF2α, ATF4, CHOP, pSTAT6, STAT6, STING, pTBK1, and actin antibodies were each added, allowed to stand at 4° C. for 16 hours, and then a secondary antibody was added and allowed to stand at room temperature for one hour. Afterward, the resulting mixture was allowed to react with an electrochemiluminescence (ECL) reagent, and the ECL was measured using a chemiluminescence image analyzer.

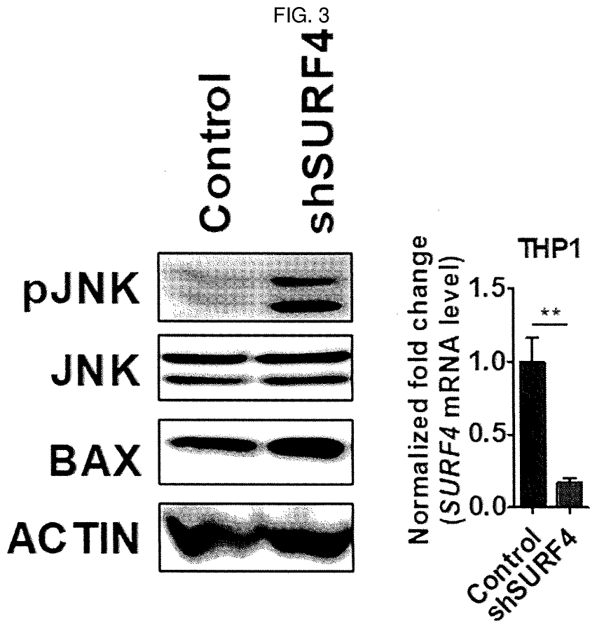

As a result, decreased expression of SURF4 increased pJNK, indicating that SURF4 is associated with the JNK pathway. It is known that the amount of BAX increases as apoptosis increases, and the results of the present experiment also confirmed that when SURF4 shRNA was transfected, apoptosis increased and BAX also increased (FIG. 3).

### (3) Measurement of Normalized Fold Change in MFI for Phosphorylated Proteins

The amount of phosphorylated proteins in HL60 and THP1 cell lines transfected with SURF4 shRNA was measured through flow cytometry. The transfected SURF4 shRNA was the same as in Example 1-(1). When measuring phosphorylated proteins, cells were collected, 1% paraformaldehyde was added, and the resulting mixture was allowed to stand at 4° C. for 30 minutes. Afterward, the mixture was washed, and 95% methanol was added, and the resulting mixture was allowed to stand at 4° C. for 30 minutes. Afterward, pJNK, pERK, and pAKT primary antibodies were added, and the cells were cultured at 4° C. for 30 minutes. A secondary antibody (Alexa Fluor 488) was added, and the cells were allowed to stand at 4° C. for 30 minutes, and then measurement was performed using a flow cytometer (FACS).

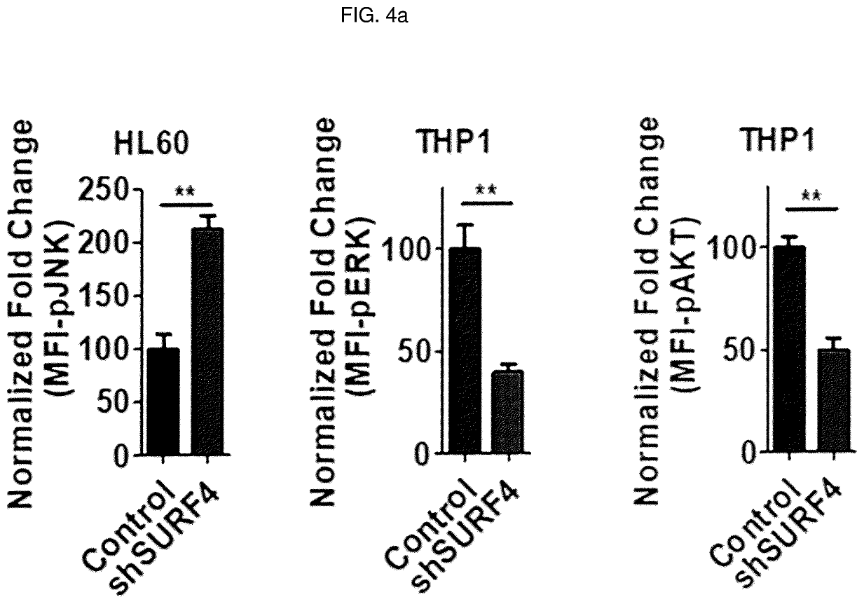

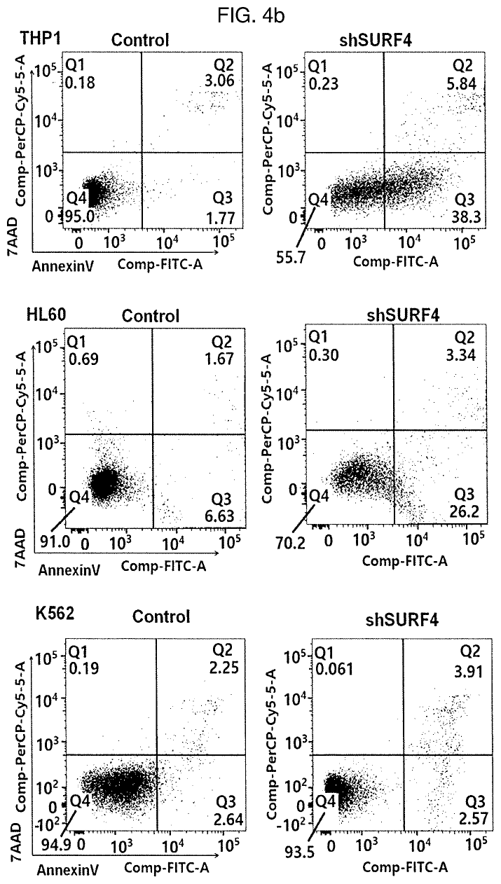

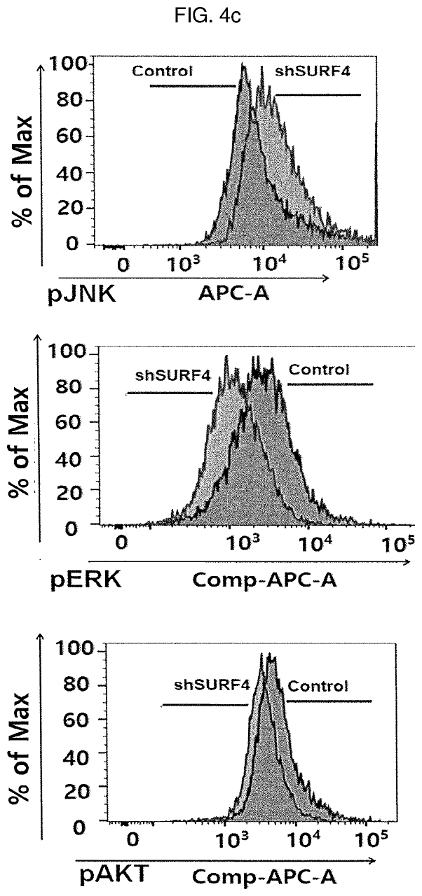

As a result, it was confirmed that there were differences in pJNK, PERK, and pAKT when the expression of SURF4 was reduced (FIG. 4A). In addition, it was confirmed that the apoptosis of THP1, HL60, and K562 cells transfected with SURF4 shRNA increased, compared to the control group, and as a result of measuring the normalized fold change in MFI for the phosphorylated proteins in THP1 and HL60 cells transfected with SURF4 shRNA, it was confirmed that the cell lines transfected with SURF4 shRNA were different from the control group (FIGS. 4B and 4C).

### Example 2: Treatment of SURF4 shRNA-Transfected Myeloid Leukemia Cells with Paclitaxel

Apoptosis was measured after treating SURF4 shRNA-transfected THP1, HL60, and K562 cells with paclitaxel. The transfected SURF4 shRNA was the same as in Example 1-(1). Specifically, the concentration of paclitaxel treated in the THP1, HL60, and K562 cells was 0.5 μM, and after transfecting the cells with SURF4 shRNA, the cells were treated with paclitaxel 7 to 10 days later. Afterward, cells of each type were collected and suspended with an Annexin V binding buffer, and then Annexin V-FITC and 7AAD were added to perform measurement using a FACS flow cytometer.

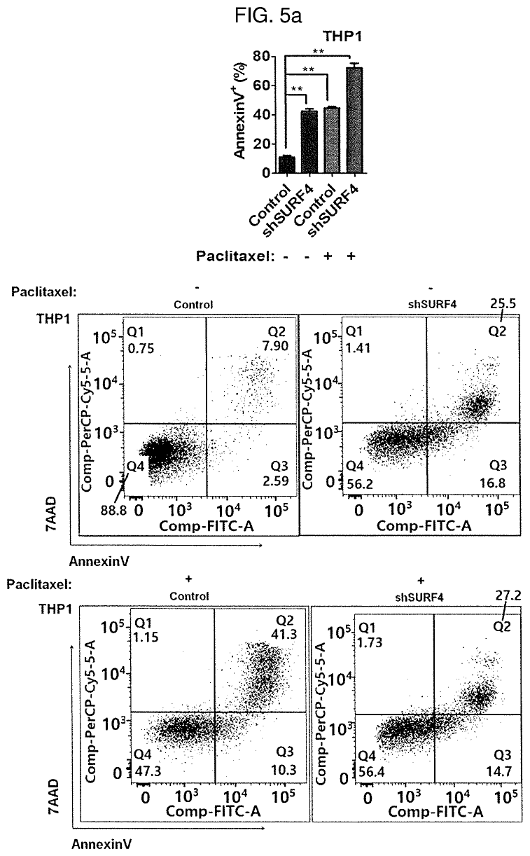

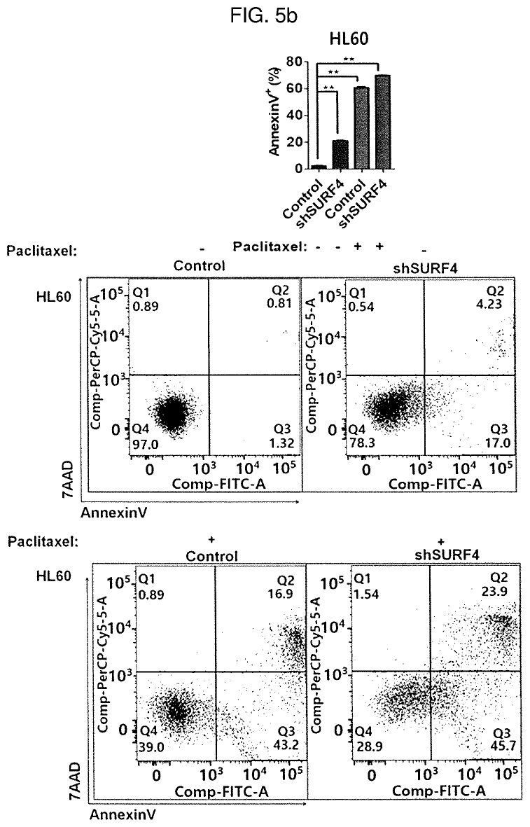

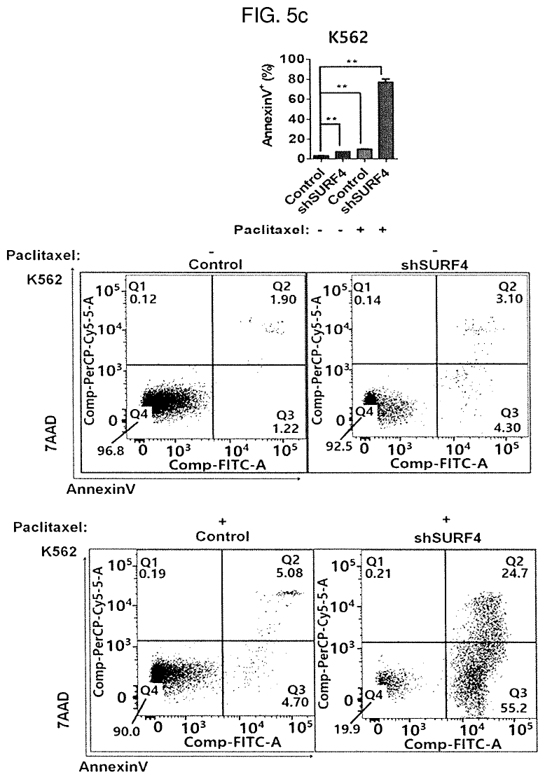

As a result, it could be seen that paclitaxel, an anticancer agent, induced apoptosis, and it could be seen that when cells transfected with SURF4 shRNA were treated with paclitaxel, apoptosis increased, compared to the case in which control cells were treated with paclitaxel alone (FIG. 5A: THP1 cells, FIG. 5B: HL60 cells, FIG. 5C: K562 cells).

In addition, Western blot was performed to confirm the expression of cleaved caspase 9, cleaved caspase 3, and PARP, which are associated with apoptosis, in THP1 cells transfected with SURF4 shRNA. The cells were lysed with a lysis buffer to extract proteins, which were then separated on a 10% SDS-PAGE gel and transferred to a membrane. pJNK, JNK, BAX, caspase 9, cleaved caspase 9, caspase 3, cleaved caspase 3, PARP, IRE1α, GRP78, PERK, pelF2α, elF2α, ATF4, CHOP, pSTAT6, STAT6, STING, pTBK1, and actin antibodies were each added, allowed to stand at 4° C. for 16 hours, and then a secondary antibody was added and allowed to stand at room temperature for one hour. Afterward, the resulting mixture was allowed to react with an ECL reagent, and the ECL was measured using a chemiluminescence image analyzer.

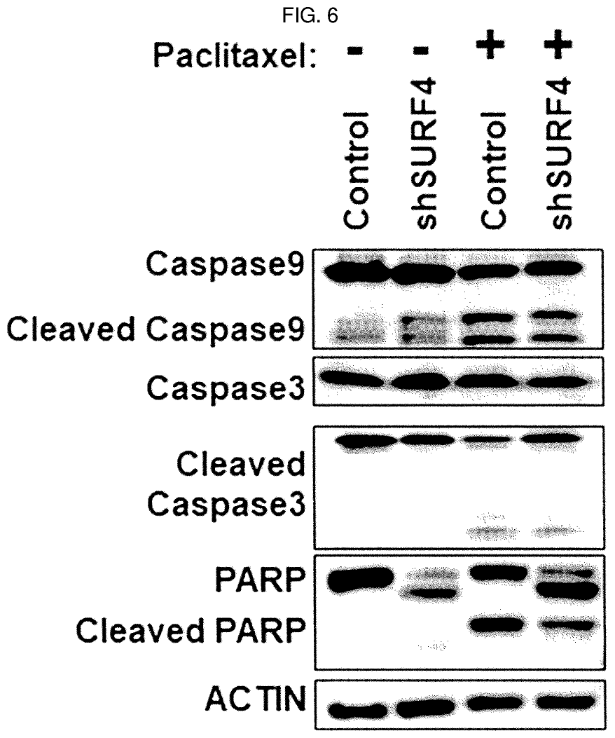

As a result, it could be seen that cleaved caspase 9, cleaved caspase 3, and PARP increased more in the cells transfected with SURF4 shRNA than in the cells treated with paclitaxel alone (FIG. 6).

Through these results, it can be seen that SURF4 and paclitaxel synergistically induce apoptosis.

### Example 3: Treatment of SURF4 shRNA-Transfected Myeloid Leukemia Cells with Tunicamycin

Apoptosis was measured after treating SURF4 shRNA-transfected THP1, HL60, and K562 cells with tunicamycin. The transfected SURF4 shRNA was the same as in Example 1-(1). Specifically, the concentration of tunicamycin treated in the THP1, HL60, and K562 cells was 10 μM, and after transfecting the cells with SURF4 shRNA, the cells were treated with tunicamycin 7 to 10 days later. Afterward, cells of each type were collected and suspended with an Annexin V binding buffer, and then Annexin V-FITC and 7AAD were added to perform measurement using a FACS flow cytometer.

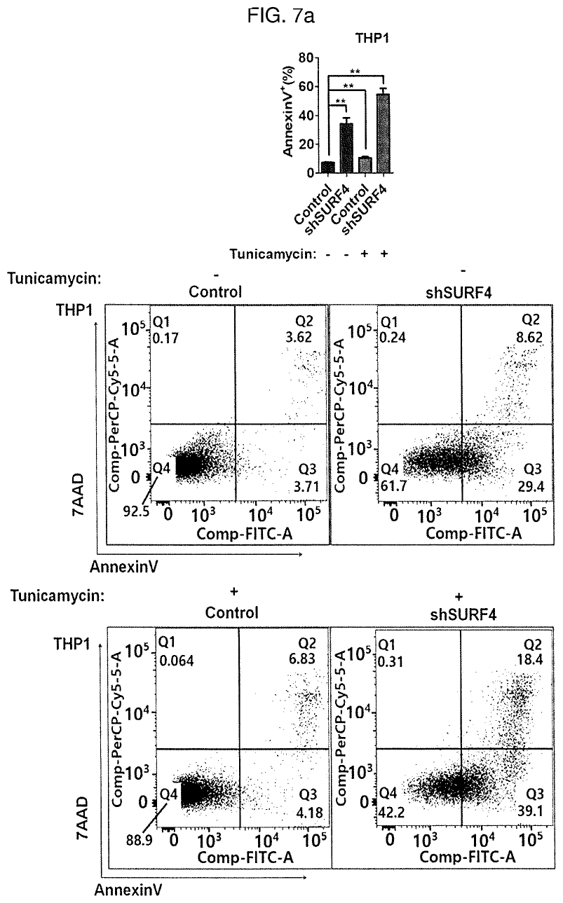

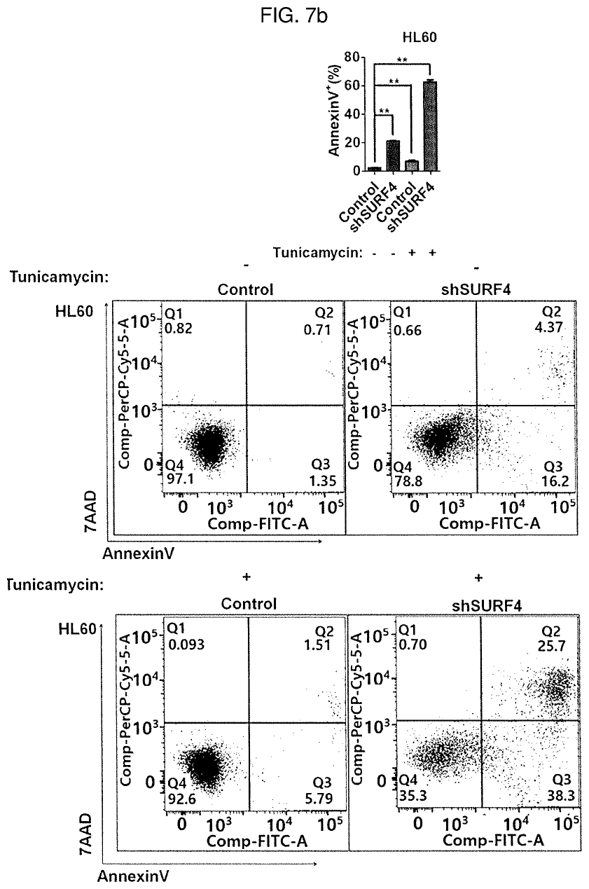

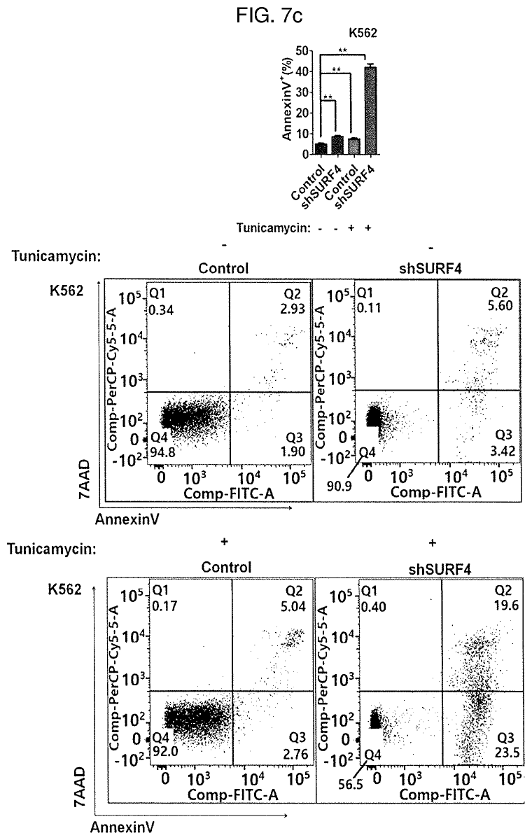

As a result, it could be seen that tunicamycin, an inducer of endoplasmic reticulum stress, induced apoptosis, and it could be seen that when cells transfected with SURF4 shRNA were treated with tunicamycin, apoptosis significantly increased, compared to the case in which control cells were treated with tunicamycin alone (FIG. 7A: THP1 cells, FIG. 7B: HL60 cells, FIG. 7C: K562 cells).

In addition, Western blot was performed to confirm the expression of PERK, pelF2α, elF2α, ATF4, and CHOP in THP1 cells transfected with SURF4 shRNA. The cells were lysed with a lysis buffer to extract proteins, which were then separated on a 10% SDS-PAGE gel and transferred to a membrane. pJNK, JNK, BAX, caspase 9, cleaved caspase 9, caspase 3, cleaved caspase 3, PARP, IRE1α, GRP78, PERK, pelF2α, elF2α, ATF4, CHOP, pSTAT6, STAT6, STING, pTBK1, and actin antibodies were each added, allowed to stand at 4° C. for 16 hours, and then a secondary antibody was added and allowed to stand at room temperature for one hour. Afterward, the resulting mixture was allowed to react with an ECL reagent, and the ECL was measured using a chemiluminescence image analyzer.

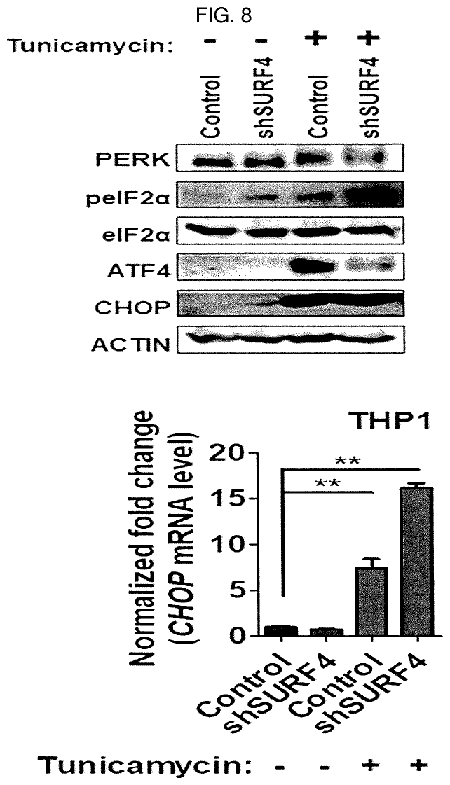

As a result, it could be seen that a PERK-pelF2α-ATF4-CHOP pathway, which operates when endoplasmic reticulum stress is induced, also increased more in the cells transfected with SURF4 shRNA than in the cells treated with tunicamycin alone (FIG. 8).

Through these results, it can be seen that when SURF4 shRNA-transfected leukemia cells are treated with tunicamycin, synergistic apoptosis is exhibited.

### Example 4: Inhibition of Growth of Myeloid Leukemia Cells Transfected with SURF4 shRNA

### (1) Quantification of Normalized Fold Change in MFI for Myeloid Differentiation (CD11b+)

Flow cytometry was performed to quantify a normalized fold change in MFI for myeloid differentiation (CD11b+) of THP1 and HL60 cells transfected with SURF4 shRNA. The transfected SURF4 shRNA was the same as in Example 1-(1), and the measurement of CD11b+ was performed by adding a CD11b+PE-Cy7 antibody to the cells, allowing the cells to stand at 4° C. for 30 minutes, and then analyzing the cells by a flow cytometer (FACS).

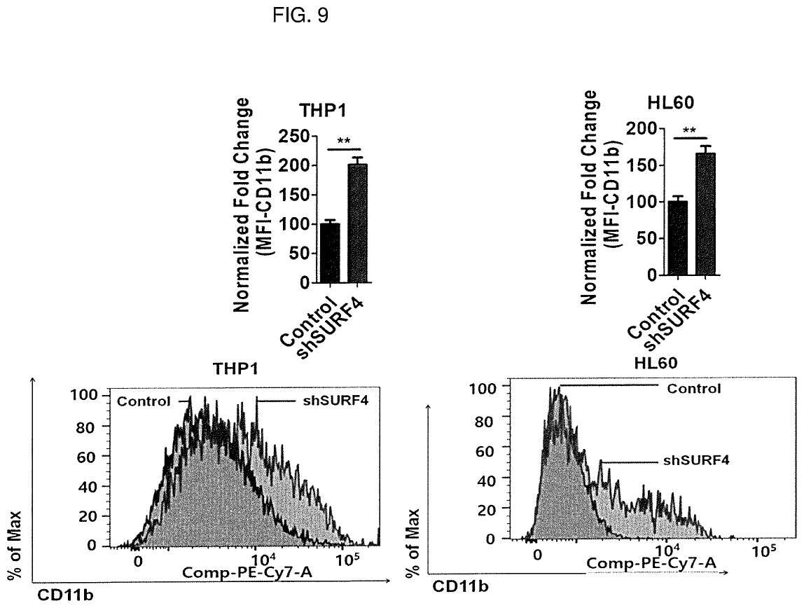

As a result, it was confirmed that when SURF4 shRNA was transfected into THP1 and HL60 cells, the amount of CD11b increased, indicating that more myeloid differentiation occurred, compared to the control group. Through this, it can be seen that myeloid differentiation increases when the expression of SURF4 is inhibited (FIG. 9).

### (2) Quantification of Normalized Fold Change in MFI for ROS

Flow cytometry was performed to quantify a normalized fold change in MFI for ROS of THP1 and HL60 cells transfected with SURF4 shRNA. The transfected SURF4 shRNA was the same as in Example 1-(1), and the measurement of ROS was performed by adding 2′,7-dichlorofluorescin diacetate (DCFDA) to the cells, allowing the cells to stand at 37° C. for 30 minutes, and then analyzing the cells by a flow cytometer (FACS).

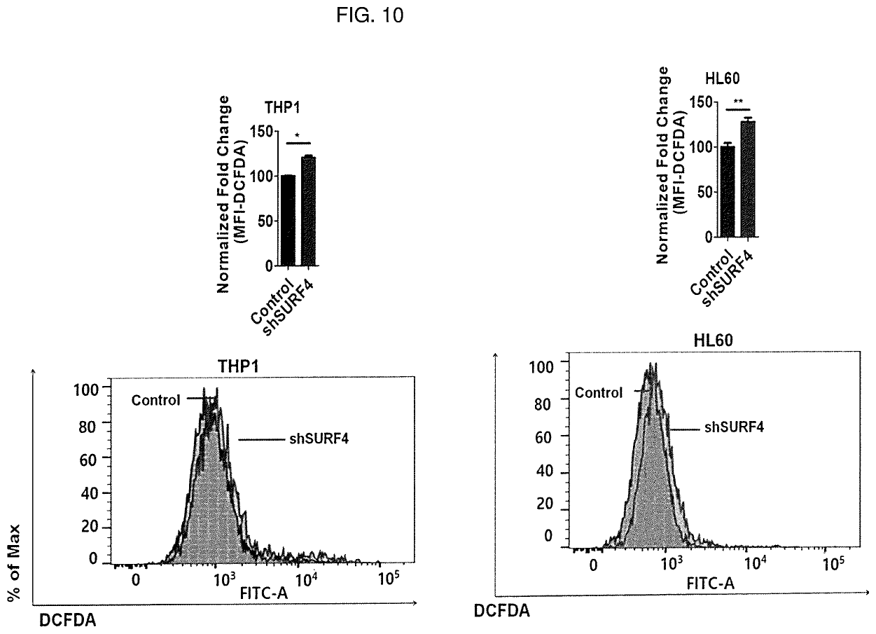

As a result, it was confirmed that when SURF4 shRNA was transfected into THP1 and HL60 cells, the amount of DCFDA, which measures the amount of ROS, increased compared to the control group. Through this, it can be seen that the amount of ROS increases when the expression of SURF4 is inhibited (FIG. 10).

### (3) Confirmation of Tumor Development in a Xenograft Model Using NOD-SCID Mice

HL60 cells transfected with SURF4 shRNA were subcutaneously injected into mice, and the tumor size was measured for 30 days. The transfected SURF4 shRNA was the same as in Example 1-(1), and 5×10⁶ HL60 cells were resuspended with 100 μl of a medium and mixed with 200 μl of VitroGel hydrogel matrix (Biogems Inc.), and 300 μl of the resulting mixture was injected subcutaneously into the mice.

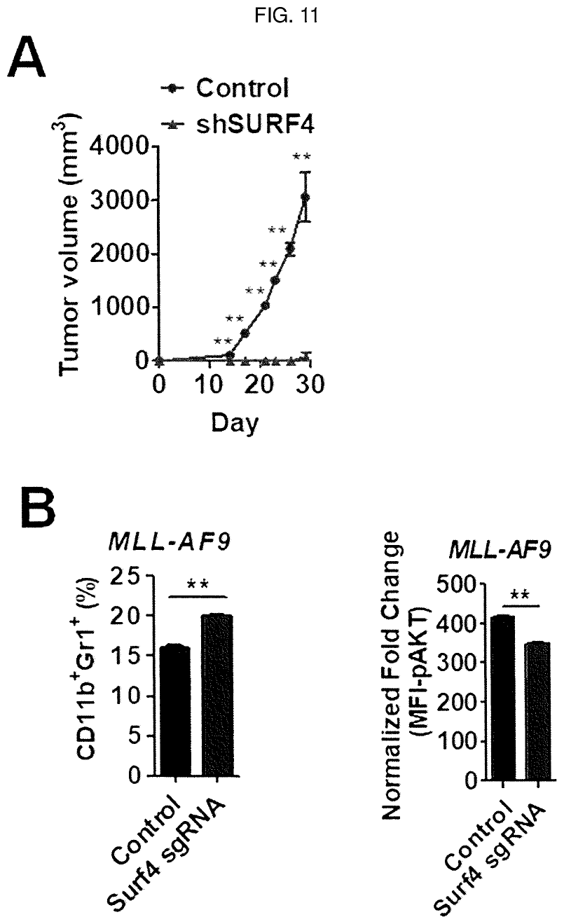

As a result, the tumor size in the control group significantly increased, compared to the case in which the cells were transfected with SURF4 shRNA were injected. In addition, it could be confirmed that when the expression of SURF4 was reduced by SURF4 shRNA in the leukemia cells prepared by transfecting the MII-Af9 gene into mouse bone marrow cells, myeloid differentiation increased, and the amount of pAKT decreased (FIG. 11).

These results show that cell growth is inhibited when the expression of SURF4 is reduced by SURF4 shRNA.

### Example 5: IL4-STAT6 and STING-Dependent Apoptosis in Myeloid Leukemia Cells Transfected with SURF4 shRNA

### (1) IL4-STAT6

Based on a report that apoptosis is induced by pSTAT6 through IL4 in leukemia cells, the expression of pSTAT6 was confirmed by treating a leukemia cell line with IL4, which induces STAT6. Specifically, THP1 and HL60 cells transfected with 6 μg of SURF4 shRNA were lysed by treating them with IL4 (5 ng/ml) for 60 minutes, and then Western blot was performed, and the SURF4 expression in the THP1 and HL60 cells transfected with SURF4 shRNA was measured by qRT-PCR. The cells were lysed with a lysis buffer to extract proteins, which were then separated on a 10% SDS-PAGE gel and transferred to a membrane. pJNK, JNK, BAX, caspase 9, cleaved caspase 9, caspase 3, cleaved caspase 3, PARP, IRE1α, GRP78, PERK, pelF2α, elF2α, ATF4, CHOP, pSTAT6, STAT6, STING, pTBK1, and actin antibodies were each added, allowed to stand at 4° C. for 16 hours, and then a secondary antibody was added and allowed to stand at room temperature for one hour. Afterward, the resulting mixture was allowed to react with an ECL reagent, and the ECL was measured using a chemiluminescence image analyzer. In addition, RNA was extracted from the cells using an RNA extraction kit, and cDNA was synthesized from the RNA using a cDNA synthesis kit. A qRT-PCR analysis was performed using the synthesized cDNA, SURF4 primers, and a SYBR green.

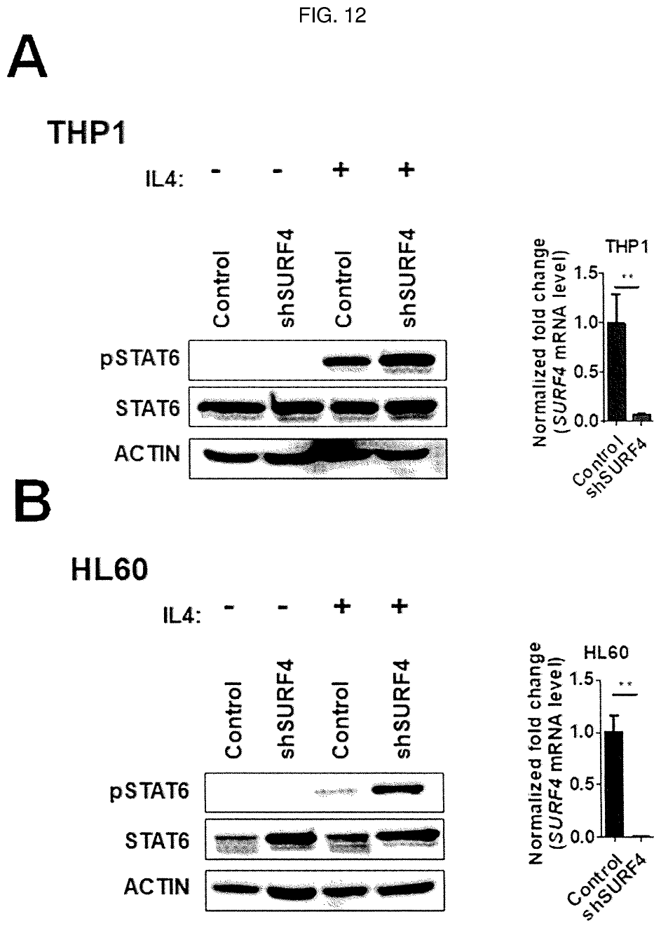

As a result of treating each of the control cells and the cells transfected with SURF4 shRNA with IL4 in the THP1 and HL60 cells, pSTAT6 increased in both the control cells and the cells transfected with SURF4 shRNA. In particular, it was confirmed that the amount of pSTAT6 increased more in the cells transfected with shRNA and treated with IL4 than in the cells of the control group treated with IL4 (FIG. 12). In addition, the normalized fold change in MFI for pSTAT6 was quantified in mouse MII-Af9 leukemia cells transfected with SURF4 shRNA.

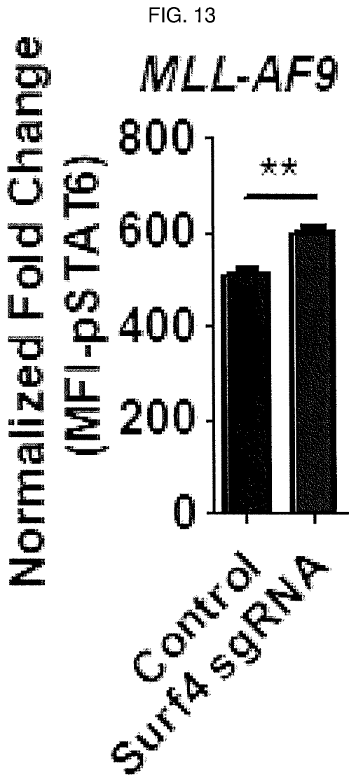

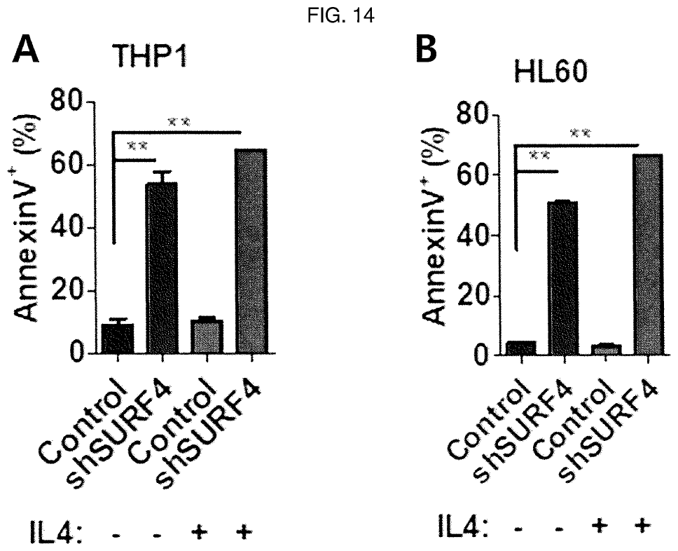

In addition, it could be seen that the amount of pSTAT6 also increased in the mouse MII-Af9 leukemia cells (FIG. 13). In addition, as a result of measuring apoptosis after treating the SURF4 shRNA-transfected THP1 and HL60 cells with IL4, it could be seen that apoptosis increased in the SURF4 shRNA-transfected leukemia cells treated with IL4, compared to the cells treated with IL4 alone (FIG. 14A: THP1 cells, FIG. 14B: HL60 cells).

Through this, it can be seen that apoptosis resulting from reduced SURF4 expression is associated with IL4-pSTAT6.

### (2) STING

Based on a report that STING, one of the endoplasmic reticulum membrane proteins, induces apoptosis and binds to SURF4 and STAT6, cells were treated with cGAMP, which induces STING. Specifically, cells were collected and lysed with a lysis buffer to extract proteins, which were then separated on a 10% SDS-PAGE gel and transferred to a membrane. pJNK, JNK, BAX, caspase 9, cleaved caspase 9, caspase 3, cleaved caspase 3, PARP, IRE1α, GRP78, PERK, pelF2α, elF2α, ATF4, CHOP, pSTAT6, STAT6, STING, pTBK1, and actin antibodies were each added, allowed to stand at 4° C. for 16 hours, and then a secondary antibody was added and allowed to stand at room temperature for one hour. Afterward, the resulting mixture was allowed to react with an ECL reagent, and the ECL was measured using a chemiluminescence image analyzer.

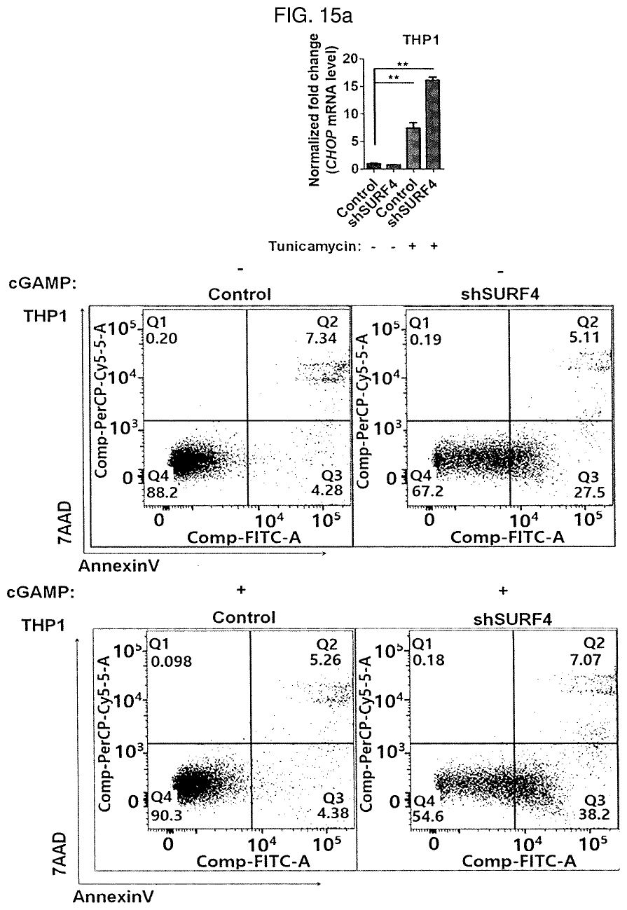

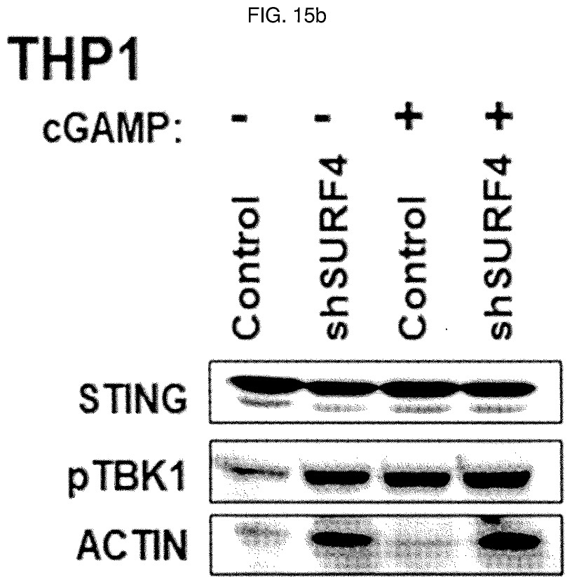

As a result, it was confirmed that when cells transfected with SURF4 shRNA were treated with cGAMP, apoptosis increased approximately two-fold (FIG. 15A), and it was confirmed that when the cells were treated with cGAMP, pTBK1 downstream of STING increased (FIG. 15B).

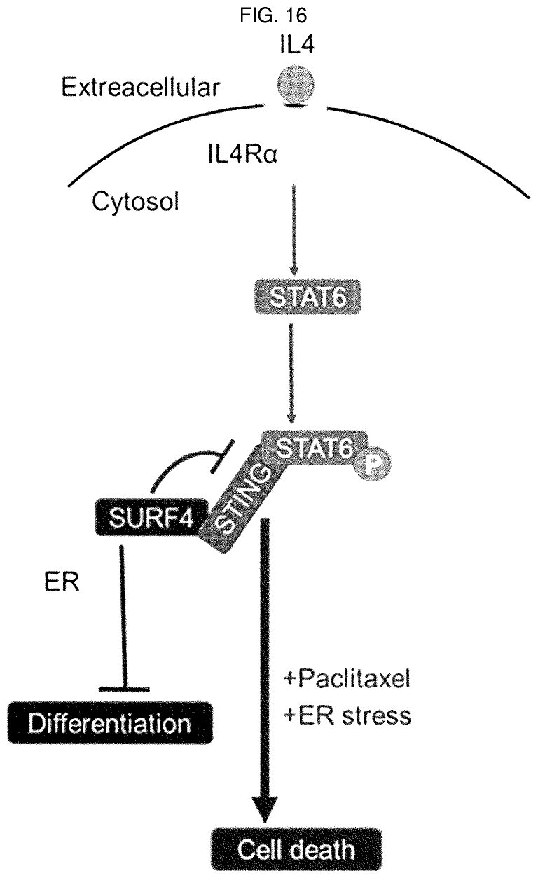

These results indicate that SURF4 regulates the functions of STAT6 and STING and inhibits the differentiation and apoptosis of myeloid leukemia cells (FIG. 16).

### Example 6: Differences in Apoptosis According to SURF4 Expression in Solid Cancer Cells

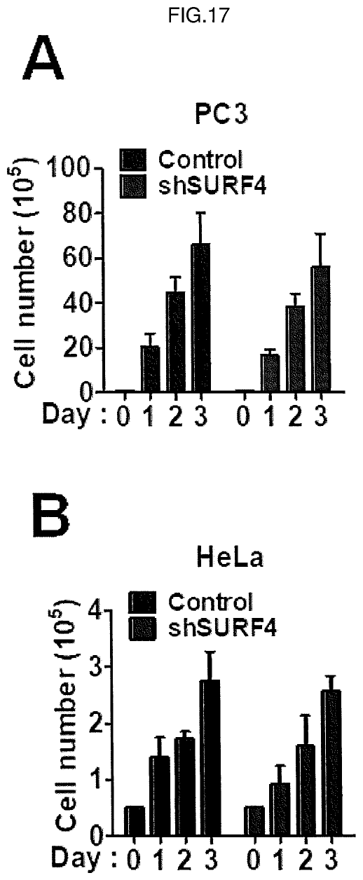

Differences in apoptosis according to the expression of SURF4 were confirmed in solid cancer cells and blood cancer cells. The cell lines used in the experiment were PC3 (prostate cancer), HeLa (cervical cancer), A2780 (ovarian cancer), MCF7 (breast cancer), and DLD1 and HCT116 (colon cancer). As a result of measuring the number of PC3 cells and Hela cells transfected with SURF4 shRNA for 3 days, there was no difference in cell growth according to the expression of SURF4 (FIG. 17A: PC3 cells, FIG. 17B: HeLa cells).

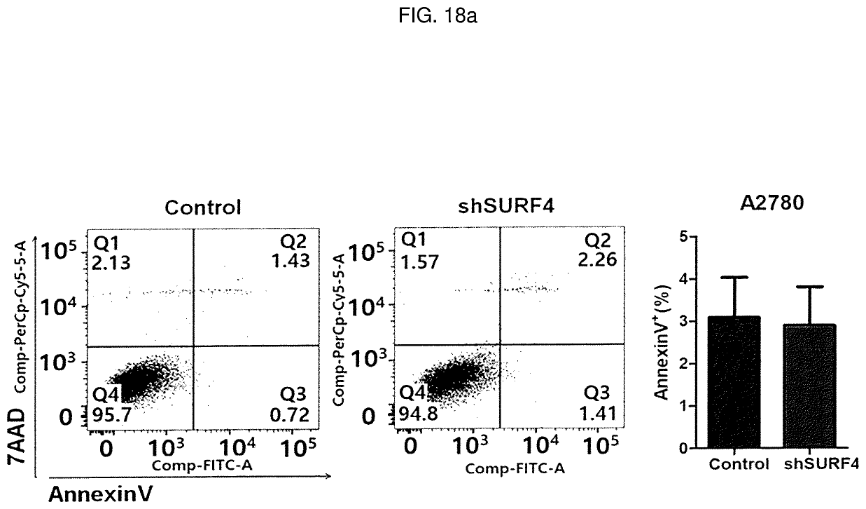

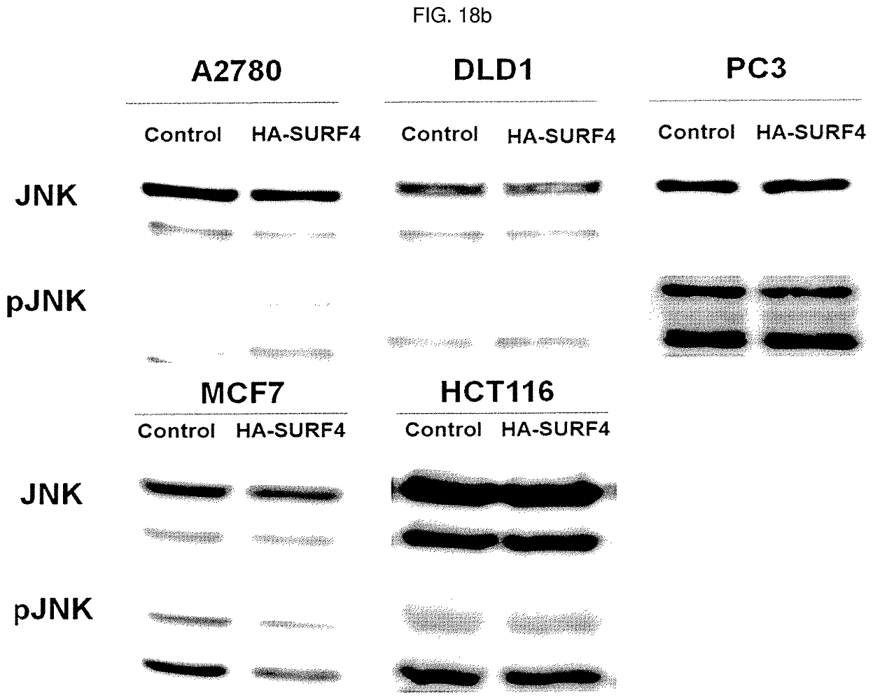

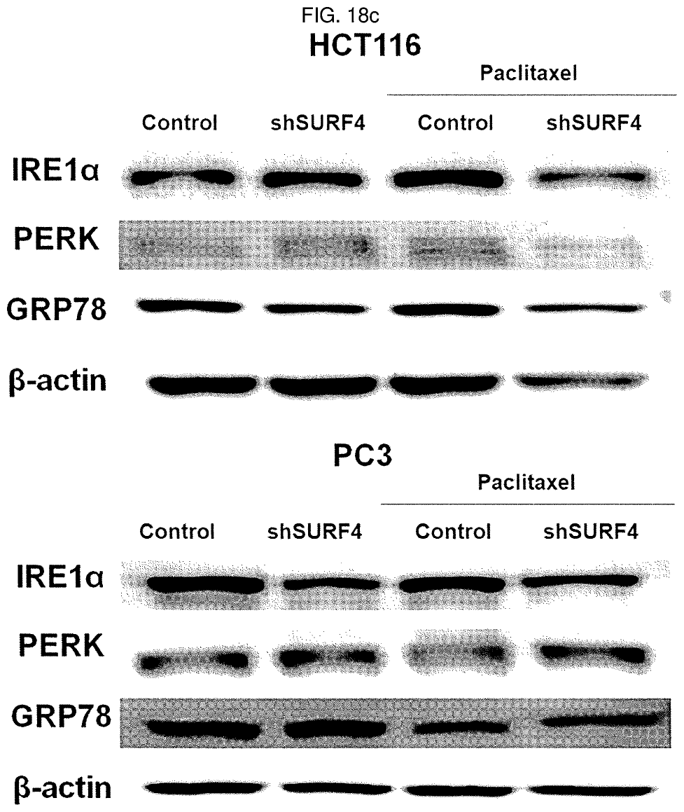

In addition, as a result of measuring apoptosis in A2780 cells transfected with 6 μg of SURF4 shRNA, it could be seen that there was no difference in apoptosis (FIG. 18A), and as a result of confirming the expression of pJNK and JNK in A2780, DLD1, PC3, MCF7, and HCT116 cells transfected with an HA-SURF4 overexpression vector by Western blot (FIG. 18B) and the expression of IRE1, PERK, and GRP78 in THP1 cells transfected with SURF4 shRNA by Western blot, it was confirmed that there was not a significant difference in the expression of pJNK and endoplasmic reticulum stress-related factors (FIG. 18C).

Through this, it can be seen that there is no difference in apoptosis according to the expression of SURF4 in solid cancer cells.

The invention claimed is:

1.-14. (canceled)

15. A method of preventing or treating blood cancer in a subject in need thereof, the method comprising administering an effective amount of a surfeit locus protein 4 (SURF4) inhibitor.

16. The method of claim 15, wherein the SURF4 inhibitor is at least one selected from the group consisting of short hairpin RNA (shRNA), small interfering RNA (siRNA), and micro RNA (miRNA).

17. The method of claim 15, wherein the SURF4 inhibitor is a culture product of cells genetically engineered to express the SURF4 inhibitor.

18. The method of claim 15, wherein the SURF4 inhibitor increases reactive oxygen species in blood cancer cells.

19. The method of claim 15, wherein the SURF4 inhibitor increases the expression of pSTAT6 in blood cancer cells.

20. The method of claim 15, wherein the SURF4 inhibitor increases the expression level of pJNK and decreases the expression levels of pERK and pAKT.

21. The method of claim 15, wherein the blood cancer is at least one selected from the group consisting of acute myeloid leukemia (AML), chronic myeloid leukemia (CML), Abelson oncogene-associated CML (Bcr-ABL translocation), myelodysplastic syndrome (MDS), B-cell acute lymphoblastic leukemia (B-ALL), T-cell acute lymphoblastic leukemia (T-ALL), chronic lymphocytic leukemia (CLL), multiple myeloma (MM), myeloproliferative neoplasms (MPN), Richter syndrome, Richter transformation of CLL, hairy cell leukemia (HCL), blastic plasmacytoid dendritic cell neoplasm (BPDCN), non-Hodgkin's lymphoma (NHL), mantle cell lymphoma (MCL), small lymphocytic lymphoma (SLL), Hodgkin's lymphoma, systemic mastocytosis, and Burkitt lymphoma.

22. The method of claim 15, further comprising an anticancer agent.

23. The method of claim 22, wherein the anticancer agent is at least one anticancer agent selected from the group consisting of paclitaxel and tunicamycin.

24. The method of claim 23, wherein when the anticancer agent includes paclitaxel, the SURF4 inhibitor further increases the expression of apoptosis-related proteins.

25. The method of claim 23, wherein when the anticancer agent includes tunicamycin, the SURF4 inhibitor further increases the expression of endoplasmic reticulum (ER) stress-related genes.
